(19) United States
(12) Patent Application Publication
(10) Pub. No.: US 2009/0317835 A1
(43) Pub. Date: Dec. 24, 2009

(54) DEVICE FOR SAMPLING CELLS BY CONTACT

(75) Inventors: Marie-Line Cosnier, Grenoble (FR); Patrice Caillat, Grenoble (FR); Franck Martin, Montpellier (FR); François Berger, Grenoble/Meylan (FR)

Correspondence Address: PEARNE & GORDON LLP, 1801 EAST 9TH STREET, SUITE 1200, CLEVELAND, OH, 44114-3108, US

(73) Assignee: COMMISSARIAT A L'ENERGIE ATOMIQUE, Paris (FR)

(21) Appl. No.: 12/307,195

(22) PCT Filed: Jul. 12, 2007
(86) PCT No.: PCT/EP07/57147

§ 371 Date: Dec. 31, 2008

(30) Foreign Application Priority Data
Jul. 13, 2006 (FR) 0652975

(51) Int. Cl. G01N 33/567; A61B 10/02

(52) U.S. Cl. 435/7.21; 600/570

(57) ABSTRACT

A microtechnological device (6) for sampling cells on or in a tissue or other element of the body. It comprises a support (12) having at least one surface of interest (14) on which is present at least one recovery zone (10) comprised of a bottom wall (22) equipped with a plurality of protuberances (24). Preferably, the microtechnological device comprises at least one recovery zone covered with a coating that has the power to attract the cells to be sampled.

### TECHNICAL FIELD

The invention relates to the field of clinical diagnostics and/or noninvasive therapeutic follow-up. More particularly, the invention relates to a microtechnological device for sampling biological cells of interest by contact.

### STATE OF THE PRIOR ART

Spatula-type tools exist for sampling cells, such as the devices for collecting cytological cells disclosed in U.S. Pat. No. 6,607,494 and in patent application PCT 99/25251. Such devices are intended to collect cells on the surface of a cell tissue directly accessible by natural routes.

If the desire is to collect cells from an organ that is not accessible directly, a biopsy is generally performed. The cells collected are then analyzed ex situ. These techniques thus alter the biological integrity of the sample. Moreover, they cannot always be used, as the insertion of a sampling device must be minimal in certain regions, such as in the brain.

Moreover, even if a biopsy or a sampling of organs from a human or an animal is performed, it is generally desirable to recover a small quantity of cells without damaging the tissue or organ sampled. Indeed, this makes it possible to proceed with other treatments or analyses of the sample taken.

### DISCLOSURE OF INVENTION

One aspect of the invention aims at mitigating the disadvantages of existing sampling devices.

One object of the present invention is notably to be able to perform noninvasive sampling of small quantities of cells.

To achieve this object, the present invention thus provides a microtechnological device for sampling cells by contact with a tissue or other element of the body comprising a support having at least one surface of interest on which is present at least one recovery zone which is formed by a bottom wall equipped with a plurality of protuberances.

Advantageously, the height of the protuberances of the inventive device is between 10 μm and 400 μm and the surface area of the protuberances is between 3×3 μm and 80×80 μm; the protuberances, for example with hexagonal or octagonal cross-sections, can be separated by gaps whose width is less than 50 μm.

It is possible that a recovery zone comprises a cup, the aforementioned bottom wall corresponding to the bottom of the cup.

According to an advantageous embodiment, the bottom wall and/or the protuberances of a recovery zone are functionalized, for example by adhesion molecules and/or antibodies, the functionalized surfaces being anionic or cationic.

In addition, the bottom wall and/or the protuberances of a recovery zone can be covered with microbeads, which can be functionalized.

The functionalization can notably include the presence of ligands bound to the surface by a silylated function.

Advantageously, the inventive device comprises a plurality of recovery zones on the same surface of interest, the recovery zones being separated by initial gap zones. Means can be present in the gap zones to separate the recovery zones, for example notches on one of the surfaces of the aforesaid support.

According to a preferred embodiment, the support of the device is in the form of plate; it comprises the first and second principal flat surfaces except for the recovery zones and the possible means to separate them.

The support can be of plastic or a microtechnological substrate, notably of silicon. It can also include a handling rod with one end joined to the support. A guide sleeve can also be provided.

Another aspect of the invention relates to a method for sampling cells on or in a tissue or other element of the body by means of a microtechnological device comprising a support having at least one surface of interest on which is present at least one recovery zone which is formed by a bottom wall equipped with a plurality of protuberances, the sampling of cells being performed by placing the aforesaid at least one recovery zone in contact with the tissue or the element of the body.

According to the invention, the sampling is not surgical in nature, that is to say, for example, that it relates to a tissue that was taken beforehand, for example by biopsy, or that it relates to a dead body. However, it is evident that the method can also be adapted and used in a living patient or animal.

In particular, the invention relates to a method for diagnosing and/or analyzing a tissue or other element of the body comprising a sampling step such as previously defined. A step of observing, by means of a microscope, the cells present on the device after sampling can be envisaged, and/or a step of culturing the cells present on the device after sampling.

### BRIEF DESCRIPTION OF DRAWINGS

The characteristics and advantages of the invention will be better understood from the description which follows and in reference to the appended drawings, which are given on a purely illustrative and non-limitative basis.

FIG. 1 represents a system for sampling by contact according to one embodiment of the invention.

FIG. 2 shows one embodiment of a recovery zone for the inventive device.

FIG. 3 illustrates an example of functionalization of the surface of the inventive device.

FIG. 4 illustrates a functionalization by beads.

FIGS. 5A to 5D illustrate various embodiments of means to separate the recovery zones of the inventive device.

FIG. 6 presents a method for using the inventive device.

### DETAILED DISCLOSURE OF INVENTION

The present invention aims at sampling a small quantity of cells from a tissue or other element of the body by means of a microtechnological sampling device described below.

In the present description, “tissue or other element of the body” means any structural or functional entity of the body of a human or animal. It is for example an organ, i.e., a differentiated functional and structural entity that is specialized for a particular function (brain, lungs, etc.). An example of a tissue is a tumor. The term “other element of the body” refers to, among other things, the skin, the cardiovascular system and the digestive system.

The sampling of cells from a tissue or other element of the body by means of a device according to the present invention can be performed in vivo or ex vivo, for example in situ.

The inventive microtechnological sampling device being of very small size, millimetric or less, said device is preferably combined with a means of handling. Various sampling systems including one such microtechnological sampling device can be envisaged according to the type of sampling planned.

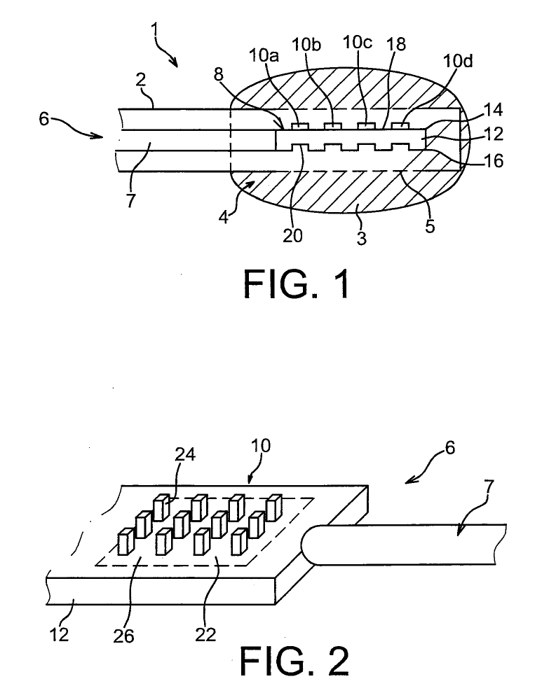

FIG. 1 illustrates an example of sampling system 1 for sampling cells within a tissue or other element of the body; the sampling can relate to a body, human or animal, living or dead, or a biopsy extracted from an animal or a patient, or any other tissue of interest. The system comprises a guide sleeve 2, for example a catheter: guide 2 makes it possible, among other things, to define the pathway of the sampling device. In particular it can be put in place beforehand, optionally under optical or radiological control, in a target zone 3 of a tissue or other element of the body. Advantageously, the extremity 4 of guide 2 is equipped with a means of obturation 5 that protects sampling device 6 during its insertion and that makes it possible to put it in contact with the tissue or other element of the body of interest 3 once it is in place. The means of obturation 5 is preferably located along the longitudinal axis of guide 2 whose distal end is closed. The means of obturation 5 can for example be a rotary or sliding window or a partially resorbable membrane.

Sampling device 6 advantageously comprises a handling rod 7 whose length depends on the use and the depth of insertion, and which can slide in guide 2. The extremity 8 of rod 7 is intended for sampling in itself. Advantageously, guide 2 and thus handling rod 7 have a very small diameter in order not to damage the tissue or other element of the body 3, and in order to allow noninvasive procedures. Thus, rod 7 can have a diameter restricted to a few millimeters, even 100 μm; guide 2 has an external diameter close to the diameter of rod 7. The rod can, for example, be of surgical stainless steel.

Extremity 8 of sampling device 6 has at least one recovery zone 10 whose developed surface is much greater than the normal surface, from three to more than twenty times greater.

Sampling device 6 thus comprises a support 12 that is preferably independent of handling rod 7 at the end at which it can be bound, for example by adhesion preferably with a biocompatible adhesive. Notably, this makes it possible to separate the manufacturing processes of the two handling and sampling parts and to use a traditional, low-cost biocompatible rod 7. Support 12 is preferably manufactured in a biocompatible material, notably silicon as specified below; the various elements comprising guide sleeve 2 are themselves compatible with a biological and/or medical use, for example made of gold or plastic, etc.

Support 12 can be of any shape, but advantageously it is flat, in the form of a plate, as it appears in the description of the manufacturing processes. In any case, support 12 can be defined with a first surface 14 and a second opposite surface 16: in the case of a support 12 that is not flat, the terms “surface” and “opposite surface” indicate portions of the external surface of support 12 that are symmetrical with respect to a cutting plane of support 12. Advantageously, surfaces 14 and 16 are comprised in a support 12 which is roughly 1 cm to 3 cm in length (in the direction of the rod) with a width of 300 μm to 800 μm and a thickness of roughly 200 μm to 400 μm.

The first surface 14 of support 12 is equipped with a recovery zone 10; it is preferable that recovery zone 10 leaves a proximal part of extremity 8 that is sufficiently long, for example 2 mm to 5 mm, to allow easy binding to rod 7. Thus, a preferred embodiment P relates to a support 12 of silicon, rectangular with dimensions of 300 μm×600 μm×2 cm, the structured zone 10 beginning at 3.2 mm from the edge bound to rod 7.

Preferably, and as illustrated, several recovery zones 10a, 10b, 10c and 10d are present on surface 14 of support 12, separated by gap zones 18. As depicted, four recovery zones are present, but it is clear that their number depends on the use, notably the size of device 6, the size of target zone 3 and the quantity of cells of interest in this zone 3, as well as the manufacturing process. Similarly, gap zones 18 can only be “virtual”, i.e., recovery zones 10 are indistinguishable a priori at the macroscopic level, but means make it possible to distinguish them at the microscopic level, even to separate them.

It is also possible that recovery zones 20 are placed on the second surface 16. Preferably, and as described more clearly below, the second recovery zones 20 are aligned and in opposition to first zones 10. The second recovery zones 20 can be identical in nature and geometry to first zones 10, or different, such as illustrated in FIG. 1: the various embodiments presented below can be combined.

According to one aspect of the inventive sampling device, at least one recovery zone of the sampling device comprises a group of protuberances or pins. As described in more detail below, the pins are intended to come in contact with the cells to be sampled, the sampled cells being trapped between the pins of the device.

FIG. 2 illustrates an example of the sampling device including a group of protuberances in a recovery zone. Recovery zone 10 comprises a bottom wall 22. According to the manufacturing process, bottom wall 22 can be placed at the bottom of an open cavity formed on the surface of support 12 or can be a surface that is “open” laterally, as represented in FIG. 2. Bottom wall 22 has a surface area s, and has a plurality of protuberances 24. Preferably, if the recovery zone comprises a cavity, the height of protuberances 24 is identical to the depth of the cavity, but it is possible that they project. In addition, it is preferable that the surface of support 12 is uniform, preferably flat, except for recovery zones 10 and the possible means of separation (described below).

Developed surface area S of recovery zone 10 is thus equal to surface are s of bottom wall 22 to which is added the surface area of each side wall of protuberances 24. According to the invention, the surface areas follow the relationship S≧3·s, the factor 3 advantageously being replaced with 5 or 10, for example.

Protuberances 24 can have any geometry desired, for example square columns or those with hexagonal cross-sections. Preferably, protuberances 24 are arranged in a regular fashion, for example in a network of square or hexagonal meshes. According to preferred embodiment P, the surface is structured in the form of octagonal pins 24 of silicon, 50 μm in height and 20 μm or 80 μm in width.

Various manufacturing processes are possible for such recovery zones 10: for example, if support 12 is of plastic, it is possible to use injection or hot embossing techniques, which make it possible to obtain by replication additional parts from molds created beforehand. Thus, protuberances 24 with 20 μm sides and a height of 50 μm can be manufactured, at low cost, on support 12 made of polyethylene, poly(methyl)methacrylate (PMMA), polycarbonate, polydimethylsiloxane (PDMS), parylene or Teflon™; one option is also to deposit one of these materials, notably parylene or Teflon™, on a plastic, metallic or other surface in order to render it biocompatible.

If a higher surface/volume ratio is desired, it is possible to use microtechnology techniques. For example, the method described in reference to FIG. 7 in document FR-A-2,846,957 can be used; however, the method described in this document is simplified because the only support 12 is machined: there is no formation of injection channels and/or sealing of the cover. Such a method makes it possible to obtain protuberances 24 with 5 μm sides and a height of 100 μm on a support 12 of silicon. More generally, protuberances 24 can have 5 μm to 20 μm sides (even sizes up to 80 μm or 100 μm can be produced in this manner) with a depth of 50 μm to 400 μm; the machining of support 12 is such that at the end of the process, the device is biocompatible. In particular, a support 12 of silicon is oxidized to be covered with SiO₂, which behaves with a biocompatibity similar to glass.

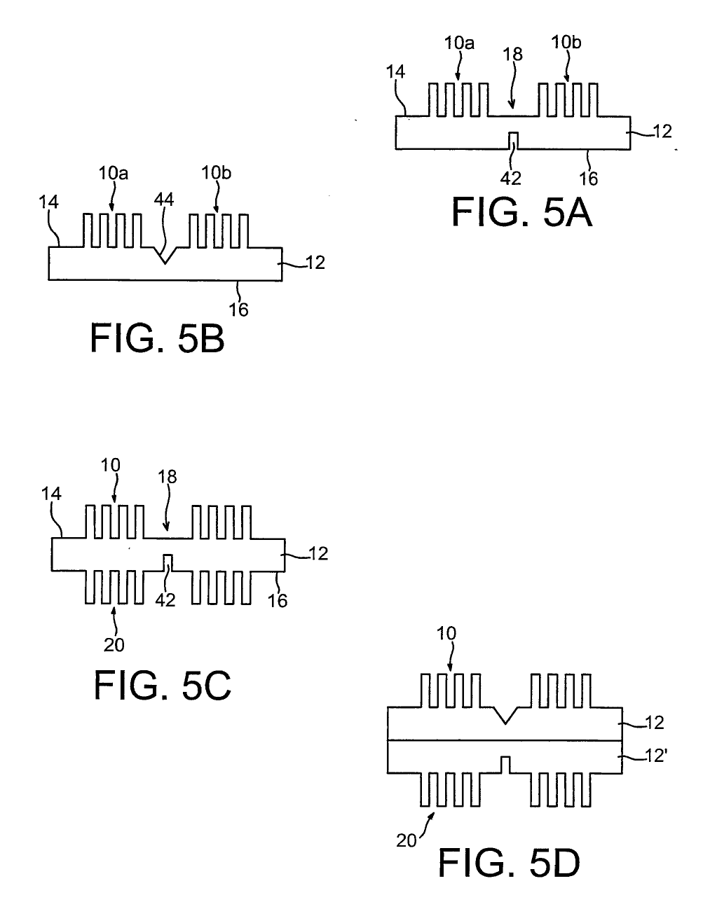

To produce recovery zones 10 and 20 on the two opposite surfaces of support 12, it is possible for example to adhere two supports, 12 and 12′, produced as above (see FIG. 5D), or to manufacture a dual surface module using microtechnology techniques on silicon or plastic molding (FIG. 5C).

Sampling cells on or in a tissue or other element of the body by means of a microtechnological sampling device such as described above consists of placing the recovery zones of the device in contact with the tissue or element of the body. This contact can be more or less pronounced. By a micro-abrasion effect, it is possible to recover cells between the protuberances or pins of the sampling device.

In order to be able to sample cells with the “lightest” contact possible, the recovery zones are preferably covered with a coating with a power of “attraction” for the cells. This power of attraction can be of electrical, chemical or physical nature, etc. Recovery zones covered with such a coating are referred to as “functionalized.”

Examples of coatings are described below. These examples encompass two families of coatings. The first family includes coatings likely to react or interact directly with cells. The second family includes coatings likely to react or interact with smaller elements, such as proteins, present around cells or in the extracellular matrix of cells. With respect to coatings of the second family, cells are attracted via the attraction between the device and the smaller target elements of the cell.

The first family notably includes coatings comprised of adhesion molecules such as adhesion proteins or peptides, coatings comprised of antibodies and anionic or cationic coatings.

The second family of coatings includes the use of DNA probes.

Another option includes the use of an electrically conductive coating possibly covered with a thin biocompatible insulating layer. Such an electrical coating can be positively or negatively polarized by means of an electrical polarization device. Such an electrical polarization device could be “on board” the sampling device, even produced as a single unit with the device.

In order to increase the power of attraction of a recovery zone, the developed surface of a recovery zone 10 also can be increased by covering bottom wall 22 and possibly protuberances 24 with microbeads. It should be noted that the microbeads should not completely fill spaces 26 between protuberances 24 in order to preserve sufficient space between the protuberances for the sampled cells.

Microbeads are commonly used in microbiology; typically they have a diameter on the order of 10 nanometers up to 100 microns and can be comprised of porous or non-porous glass, which enables them to be functionalized and to remain biocompatible.

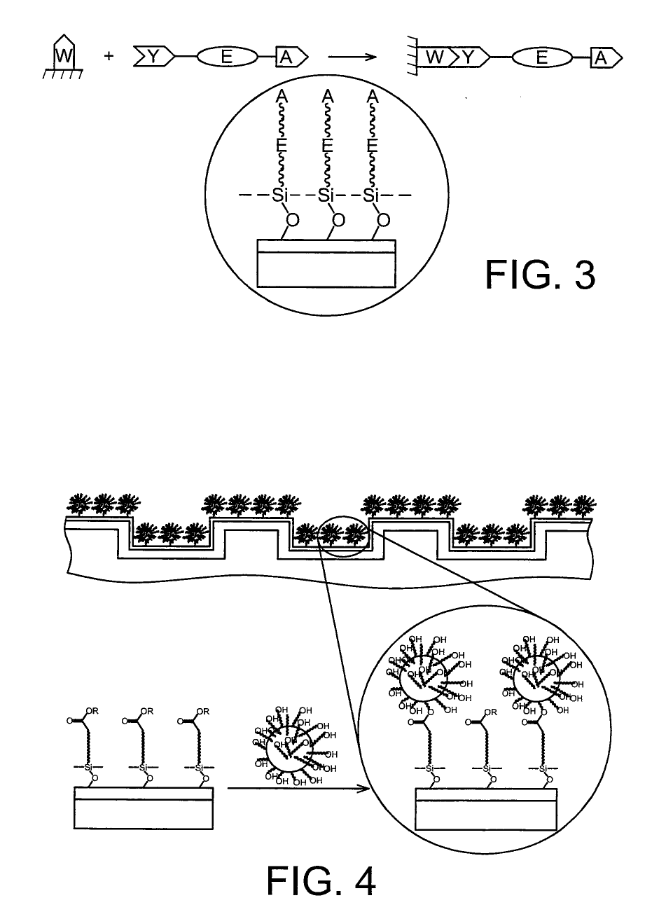

An example of functionalization of a recovery zone is described in relation to FIG. 3. The method of functionalization is carried out into two or three steps.
1) Synthesis of a bifunctional organic molecule Y-E-A, called a coupling agent, which allows non-covalent interfacial adhesion between proteins and the organic support.
2) Fixation of the coupling agent on inorganic support 12, which can be treated beforehand to present a coupling function W (in particular O—Si silylated function W for substrates 12 of silicon covered with a coating of silicon dioxide), by reaction of one of the two functions Y with the surface, the other function A reacting with the protein by forming a non-covalent bond.
3) If terminal function A allowing adsorption of the protein could not be synthesized with the silylated function due to chemical incompatibility, the modified support will undergo one or more post-silanization reactions until the latter is obtained.

Coupling function A corresponds to all existing organic and mineral functions such as: CH₃, alkenes, alkynes, aryl derivatives, halogens (Br, Cl, I, F), organometallic derivatives, alcohols, phenols, diols, ethers, epoxides, carbonyl derivatives (aldehydes, ketones, carboxylic acids, carboxylates, esters, amides, acid chlorides, acid anhydrides), nitrogen derivatives (amines, nitrated derivatives, diazo derivatives, imines, enamines, oximes, nitrites), phosphorous derivatives (phosphines, phosphites, phosphates, phosphonates), silicon derivatives, sulfur derivatives (sulfides, disulfides, thiols, thioethers, sulfones, sulfites, sulfates, sulfonic acids, sulfonates, azasulfoniums), selenium derivatives, etc.

A spacer group E, used between the two functions A and Y of the coupling agent, makes it possible to confer particular properties on the film obtained by silanization. Group E is selected among the radicals that make it possible to obtain an organized monolayer: a long-chain alkylene radical E allows an inter-chain interaction (particularly preferred among alkylene radicals E are those with 8 to 24 carbon atoms); a radical E comprising two triple bonds —C≡C— allows a cross-linking; a radical E comprising a conjugated aromatic chain (phenylene-vinylene and phenylene-acetylene radicals, for example) confers nonlinear optical properties; a pyrrole, thiophene or polysilane radical E confers electron conduction; a heterosubstituted polyaromatic radical E (quinones and diazo compounds, for example) confers properties of photo/electroluminescence; an alkyl or fluoroalkyl group E, notably an alkyl or fluoroalkyl group with 3 to 24 carbon atoms, makes it possible to use layers obtained with chromatography or electrophoresis.

Regarding the functionalization of beads, the same principle is used.

In addition, it is possible to deposit various types of beads according to sampling zones 10i and thus to obtain a tool presenting a range of affinity functions A.

For example, for a substrate 12 with a hydrophilic surface such as SiO₂, silanized, the surface ester functions located on the tool will react with functionalized beads carrying a primary hydroxyl function. After immobilization of the beads, the tool has a hydrophilic developed surface (FIG. 4).

A sampling device according to the present invention makes it possible by virtue of the pins present on the cell recovery zones to recover one or more layers of cells of the tissue or other element of the body of interest. In the case of pins of a few tens of microns in height, it is possible to recover up to ten cell layers, depending on the size of the cells collected.

Once cell sampling is performed, it is possible to analyze the spatial composition of the layers of sampled cells. One can for example use a microscope to visually identify the nature of the sampled cells.

An advantage of the sampling device according to the present invention is that the presence of multiple layers of cells makes it possible to obtain a histological section of a tissue or other element of the body.

After sampling, the recovered cells can be cultured in order to have available a greater number of cells.

From the sampled and/or cultivated cells, it is possible to perform genomic or transcriptomic analyses.

In addition, a sampling device according to the present invention makes it possible to recover “aggregates” of cells and not “dissociated” cells. Therefore, the culturing of cell aggregates makes it possible to obtain better yields and a better quality of the cultivated cells. It is thus possible to envisage the practice of cellular therapies from cells, notably cell aggregates, sampled by a device according to the present invention.

For example, one such therapy could consist of taking “sick” autologous cells, amplifying them (notably by culture), modifying their genome and then reintroducing them in the tissue or other element of the body sought to be cured.

In addition, from cells taken by means of a device according to the present invention, it is possible to perform various analyses of the molecules present in the cells or generated therefrom.

If the recovery zones are covered with a coating of the second family, the cells present between the protuberances of a recovery zone can also be eliminated and then the molecules “trapped” by the coating can be analyzed.

In addition, if sampling device 6 has several recovery zones 10i (see FIG. 1), it is possible to use the same functions on each recovery zone, or to perform a spatial differentiation, such as for example by the localized deposition of droplets (“spotting”) known for DNA chips.

According to the use and notably the analyses of the sampled molecules, it may be desirable to separate recovery zones 10a-10d of the same device 6. In particular, supports 12 can be sectioned for each preceding embodiment.

In order to facilitate the sectioning of support 12, advantageously, gap zones 18 between recovery zones 10 are equipped with means of separation. For example, notches can be etched when protuberances 24 and/or the walls are produced: FIG. 5. Gap zones 18 can then be sectioned easily.

Various embodiments are possible: for example, it is possible to produce a sectioning primer 42 by the etching of support 12 on surface 16 opposite to surface 14 comprising recovery zones 10, by masking and etching for example (FIG. 5A). This notch 44 can also be produced on “forward” surface 14, or choose for example isotropic chemical engraving, for example with KOH (FIG. 5B).

If recovery zones 10 and 20 are present on each surface 14 and 16, it is possible to position the means of separation on only one of the surfaces (FIG. 5C), or on both (FIG. 5D). In this respect, two embodiments should be noted for devices comprising recovery zones 10 and 20 on each of their opposite surfaces 14 and 16: one support 12 (FIG. 5C) or two joined supports 12 and 12′ (FIG. 5D).

It is also clear that the various embodiments of notches 42 and 44 can be used interchangeably and in combination.

According to preferred embodiment P, a silicon wafer 100 mm in diameter is machined to obtain a number of one hundred and forty two final devices after cutting. Support 12 of silicon is advantageously marked: notably, the name of the device, alignment crosses and cutting marks, etc., are etched, for example at 500 nm, by photolithography with masking and dry etching.

The reverse surface undergoes a similar treatment (photolithography with masking aligned with the previous, 5 μm to 10 μm dry etching, shrinking of the mask resin) to form notches. The forward surface is then drawn and etched for microstructuring, with photolithography with aligned mask, dry etching at a depth of 50 μm and shrinking of the resin. The surfaces are then prepared in order to allow their biological and/or medical use: in particular the polymer (for example C₄F₈) formed on the flanks of the cavities during etching is eliminated, by complete deoxidation, followed by wet oxidation at 100 nm, then total deoxidation; a final layer of SiO₂ is obtained by wet oxidation at 500 nm.

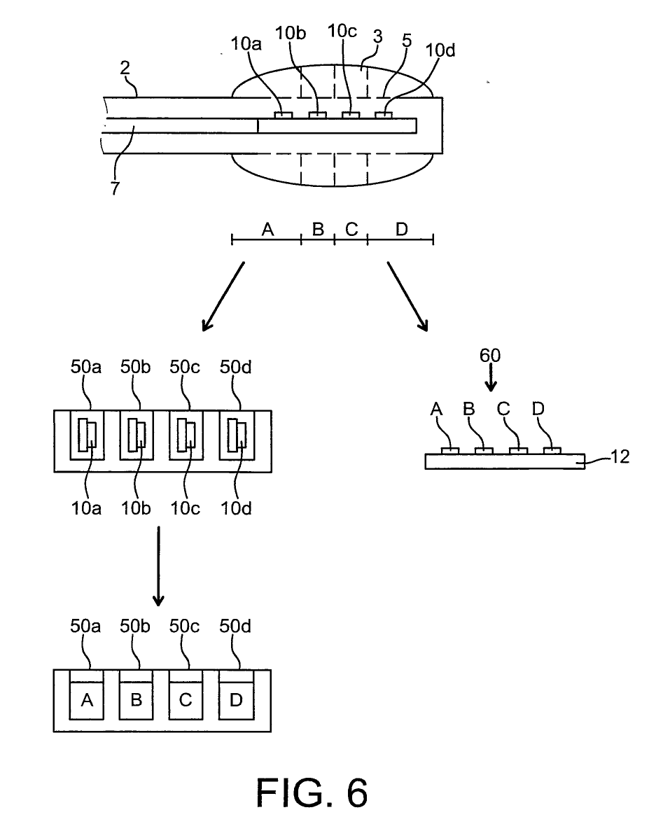

According to a mode of use of the inventive device illustrated in FIG. 6, guide 2 is first put in place, preferably under control in target zone 3; support 12 is adhered to the end of rod 7. Rod 7 is inserted in guide 2, also under optical control in order to ensure the precision of its positioning, and in particular to determine zones A, B, C and D of tumor 3 corresponding to each recovery zone 10a-10d. Once recovery zones 10a-10d are in place, the means of obturation 5 are opened, and the sampling is performed by apposition; no manipulation of the device itself is necessary, the contact surface of recovery zones 10 being directly accessible (no cover, for example). This also allows the unit, and notably support 12, to be miniaturized. The means of obturation 5 can then optionally be closed. Rod 7 is then withdrawn from guide 2, support 12 is detached therefrom, and recovery zones 10a-10d can be analyzed.

Two approaches to treating the sample taken can be implemented during the analysis:

1) Device 6 is divisible, and each zone 10a-10d is treated independently. In fact, once rod 7 is withdrawn, support 12 is broken and the various zones 10a-10d can be treated and analyzed separately. For example, cells A, B, C and D are extracted and then introduced into tubes 50a-50d and “rinsed” with solutions that extract the cells from the recovery zones.

2) It is also possible not to divide support 12, which thus preserves the definition of active zones A-D staged within tumor 3. It is support 12 itself that serves as the substrate for the final analysis of device 60, for example for observation under a microscope or for culturing the cells.

Whichever approach is chosen, a mapping of the tissue of interest, and results concerning cellular and protein composition as a function of depth in target zone 3, can be obtained.

The inventive sampling device thus exhibits the following particularly advantageous characteristics:
sampling is relatively non-invasive: in particular, the diameter of device 6, and even of system 1, is reduced, notably to a few millimeters, preferably 1 mm,
sampling is relatively non-aggressive: it is achieved by contact (or “apposition”) without sectioning tissue 3,
the machined part actually used for sampling is reduced and only encompasses support 12, which can be combined with a low-cost handling rod 7,
machining of the part used for sampling 12 is reduced to the manufacture of contact zones 10 and 20, with no other mechanical elements or additional steps of sealing or joining,
device 6 can be used in vivo or post-operative procedures, or used in vitro on a sampled tissue, and serve as the basis for cellular and possibly molecular analysis,
the presence of staged recovery zones 10a-10d makes it possible to analyze after printing the distribution of the cells of interest in sampling zone 3,
each recovery zone 10 can be functionalized according to the cells targeted and/or the type of subsequent analysis or treatment,
each recovery zone 10a-10d can be separated from the others and analyzed by its own technique,
device support 12 can be compatible with any equipment used for subsequent analysis or treatment,
a mapping according to the analyzed axis of depth of zone 3 can be established according to successive and differentiated active zones A-D along the device; proceeding under stereoscopy indeed makes it possible to precisely guide device 6 and to know exactly which region A-D was probed.

### Example Embodiment

An example of anionic coating is described below.

Preceding device P (support 12 in Si of 600×300 μm², with octagonal protuberances 24) was silanized and then functionalized to give the carboxylate function. Indeed, at physiological pH, biological systems are naturally charged; ionic interactions (based on the principles of chromatography) can be used to specifically absorb protein markers. For anionic (negatively charged) surfaces, carboxylate derivatives are most commonly used.

Since carboxylate and silane functions are incompatible, a strategy of indirect synthesis via trimethoxysilylundecan-10-oic acid methyl ester was selected.

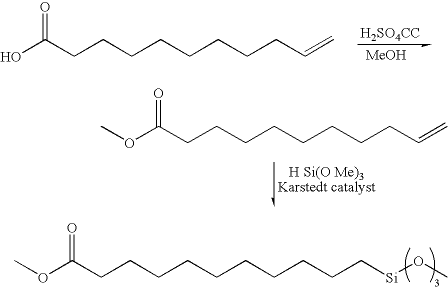

The acid function is protected in the form of a methyl ester after reaction of undecenoic acid with sulfuric acid and methanol; incorporation of the silylated group is performed classically by a hydrosilylation reaction.

For example, 10-undec-1-enoic acid methyl ester is manufactured to form trimethoxysilylundecan-10-oic acid methyl ester by the following process:
Concentrated sulfuric acid (12.88 g; 7 ml; 131 mmol; 2.3 eq) is added to a solution of undecenoic acid (98%) (10.47 g; 11.5 ml; 56 mmol) dissolved in 500 ml of methanol. The reaction proceeds at 0° C. for 4 hours.
After the methanol evaporates and the ethyl acetate is taken up, the reaction mixture is washed successively with EDI (×2) and with a saturated sodium chloride solution, dried on anhydrous magnesium sulfate and then concentrated to yield a colorless liquid (10.99 g; 99%). The following characteristics are obtained:

### δ_{H}(200 MHz; CDCl₃): 1.30 (10H; m; H⁵⁻⁹)

1.62 (2H; m; H⁴)
2.04 (2H; m; H¹⁰)
2.31 (2H; t; H³; ³J_{H-H}=7.4 Hz)
3.67 (3H; s; H¹)
4.97 (2H; m; H¹²)
5.81 (1H; m; H¹¹)
   δ_{c}(200 MHz; CDCl₃): 25.31
29.26
29.42
29.50
29.58
29.66
34.16
34.44
51.76 (C¹)
114.51 (C¹²)
139.46 (C¹¹)
174.61 (C²)
10-undec-1-enoic acid methyl ester (10.58 g; 53 mmol) is mixed with trimethoxysilane (95%) (8.75 g; 9.1 ml; 68 mmol; 1.3 eq). Karstedt catalyst (0.13 g; 0.13 mmol; 0.0025 eq.) is added very slowly. The reaction proceeds at room temperature for 16 hours. The crude reaction product is purified by distillation to yield a colorless liquid (120-125° C. at 0.5 mbar; 11.7 g; 70%):

### δ_{H}(200 MHz; CDCl₃): 0.65 (2H; m; H¹²)

1.27 (14H; m; H⁵⁻¹¹)
1.62 (2H; m; H⁴)
2.30 (2H; t; H³; ³J_{H-H}=7.4 Hz)
3.57 (9H; s; H¹³)
3.67 (3H; s; H¹)
   δ_{c}(200 MHz; CDCl₃): 9.21 (C¹²)
22.68
25.04
29.23
29.38 (2C)
29.50 (2C)
33.17
34.19
50.55 (C¹³)
51.46 (C¹)
174.38 (C²)

### δ_{Si}(200 MHz; CDCl₃): −41.30 (s)

Hydroxylation of the silicon substrate covered with a 500 nm thermal oxide coating is performed in a 3.5 M soda solution for 2 hours, with a 10⁻² M silanization solution in anhydrous trichloroethylene, the silanization reactions being performed at a controlled temperature of 2° C. for 24 hours.

The modified support is placed in contact with an aluminum iodide solution in order to release the carboxylic acid function, which in turn reacts with an aqueous soda solution to yield the corresponding carboxylate function.

Those skilled in the art will be able to imagine other embodiments of a sampling device according to the present invention as well as other uses of one such device.

1-26. (canceled)

27. A microtechnological device for sampling cells by contact with a tissue or other element of the body comprising a support having at least one surface of interest on which is present at least one recovery zone which is formed by a bottom wall equipped with a plurality of protuberances.

28. Device according to claim 27, wherein the bottom wall and/or the protuberances of a recovery zone are functionalized.

29. Device according to claim 28, wherein the functionalization is achieved by adhesion molecules.

30. Device according to claim 28, wherein the functionalization is achieved by antibodies.

31. Device according to claim 28, wherein the functionalized surfaces are anionic or cationic.

32. Device according to claim 27, wherein at least one recovery zone comprises a cup, the aforementioned bottom wall corresponding to the bottom of the cup.

33. Device according to claim 27, comprising a plurality of recovery zones on the same surface of interest, the recovery zones being separated by initial gap zones.

34. Device according to claim 33, comprising means to separate the recovery zones in the gap zones.

35. Device according to claim 33, wherein the means to separate comprise notches on one of the surfaces of the aforesaid support.

36. Device according to claim 27, wherein the support is in the form of plate.

37. Device according to claim 36, wherein the support comprises the first and second principal flat surfaces except for the recovery zones and the possible means to separate them.

38. Device according to claim 27, wherein the support is of plastic.

39. Device according to claim 27, wherein the support is a microtechnological substrate, notably of silicon.

40. Device according to claim 27, wherein the height of the protuberances is between 10 μm and 400 μm and the surface area of the protuberances is between 3×3 μm and 80×80 μm.

41. Device according to claim 27, wherein the protuberances are separated by spaces whose width is less than 50 μm.

42. Device according to claim 27, wherein the protuberances have hexagonal or octagonal cross-sections.

43. Device according to claim 27, wherein the bottom wall and/or the protuberances of a recovery zone are covered with microbeads.

44. Device according to claim 43, wherein the microbeads are functionalized.

45. Device according to one of the claims 28, 29, 30, 31 or 44, wherein the functionalization comprises the presence of ligands bound to the surface by a silylated function.

46. Device according to claim 27, comprising in addition a handling rod, the support being able to be attached to one extremity of the rod.

47. Sampling system comprising a device according to claim 46 and a guide sleeve.

48. Method for sampling cells on or in a tissue or other element of the body by means of a microtechnological device comprising a support having at least one surface of interest on which is present at least one recovery zone comprised of a bottom wall equipped with a plurality of protuberances, cell sampling being performed by placing the aforesaid at least one recovery zone in contact with the tissue or element of the body.

49. Method of diagnosis comprising a sampling step according to claim 48.

50. Method for analyzing a tissue or other element of the body comprising a sampling step according to claim 48.

51. Method according to claim 48, comprising a step of observing, by means of a microscope, the cells present on the device after sampling.

52. Method according to claim 48, comprising a step of culturing the cells present on the device after sampling.
